(19) United States
(12) Patent Application Publication
(10) Pub. No.: US 2011/0182851 A1
(43) Pub. Date: Jul. 28, 2011

(54) OXIDIZED LDL SPECIFIC ANTIBODY-FUSION AND CONJUGATED PROTEINS

(76) Inventor: Jan Nilsson, Genarp (SE)

(21) Appl. No.: 13/003,648

(22) PCT Filed: Jul. 13, 2009
(86) PCT No.: PCT/SE2009/050896

§ 371 Date: Jan. 11, 2011

(30) Foreign Application Priority Data
Jul. 11, 2008 (SE) 0801665-1

(51) Int. Cl. A61K 38/20; C07K 19/00; A61K 39/395; A61P 9/10

(52) U.S. Cl. 424/85.2; 530/391.7; 530/351; 424/178.1

(57) ABSTRACT

The present invention relates to complete oxidized LDL specific IgG fused or conjugated with at least one of the proteins of the group IL-10, TIMPs, and TGFβs to be used in a medicine, the use thereof for treatment of atherosclerosis and prevention of clinical events in patients with atherosclerosis, pharmaceutical compositions containing the same, as well as method for treatment of atherosclerosis and prevention of clinical events in patients with atherosclerosis.

### TECHNICAL FIELD

Atherosclerosis is the major cause of acute myocardial infarction and stroke. The disease is characterized by chronic inflammation of the arterial intima [1]. Several lines of evidence have demonstrated that this inflammation is caused by accumulation and subsequent oxidation of low-density lipoprotein (LDL) particles in the arterial extracellular matrix. LDL oxidation is associated with formation of a number of reactive aldehydes, phospholipids and other lipid derivates that interact directly with pro-inflammatory signal pathways or cause toxic injury to surrounding cells [2]. The combined toxic and pro-inflammatory effect of oxidized LDL results in development of chronically inflamed scar on the inside of the vessel—an atherosclerotic plaque. The atherosclerotic plaque is generally clinically silent until the continues eroding effect of oxidized LDL results in breakdown of the plaque filamentous structures, rupture of the plaque cap and formation of an occluding thrombus that blocks oxygen supply to distal tissues for example in the myocardium. Plaque erosion is caused by release of pro-inflammatory cytokines and matrix-degrading matrix metallo-proteinases (MMPs) from cells exposed to oxidized LDL. Other cells protect against these processes by releasing anti-inflammatory cytokines such as interleukin (IL-10), matrix stabilizers such as transforming growth factor (TGF)β and inhibitors of MMPs such as tissue inhibitor of matrix metallo-proteinases TIMP. The balance between these two processes determines if the plaque will remain stable and clinically silent or became unstable and give rise to an acute cardiovascular event[1].

Oxidized LDL is also taken up by antigen presenting cells leading to induction of adaptive immune responses against antigens in oxidized LDL [4, 5].

Immunization of hypercholesterolemic animals with oxidized LDL is associated with increased levels of oxidized LDL-specific IgG and inhibition of atherosclerosis demonstrating the existence of adaptive athero-protective immunity [6-9]. Following detailed mapping of oxidized LDL antigens we have identified a number of native and aldehyde-modified peptide sequences in the LDL protein apoB-100 as targets for these immune responses [10] and demonstrated that immunization of apo E^{-/-} mice with the corresponding synthetic peptides significantly reduces the development of atherosclerosis [11-13]. A particularly strong protective effect was seen in mice immunized with the apoB-100 peptide #45 (amino acids 661-680) [13]. Interestingly, high levels of IgG autoantbodies against this apoB-100 peptide was found to be associated with a lower risk for development of acute cardiovascular events in a subsequent prospective epidemiological study [14]. We have developed human recombinant IgG1 antibodies specific for aldehyde-modified #45 apoB-100 sequence (BI-204 or 2D03) which bind to oxidized but not to native LDL. Treatment of apo E^{-/-} mice with this antibody reduced atherosclerosis by almost 50% over a 4-week period [15]. An even more dramatic effect of the antibody treatment was observed in LDL receptor^{-/-} mice carrying the human gene for apoB-100 [16] or the full length mouse apoB-100 [17].

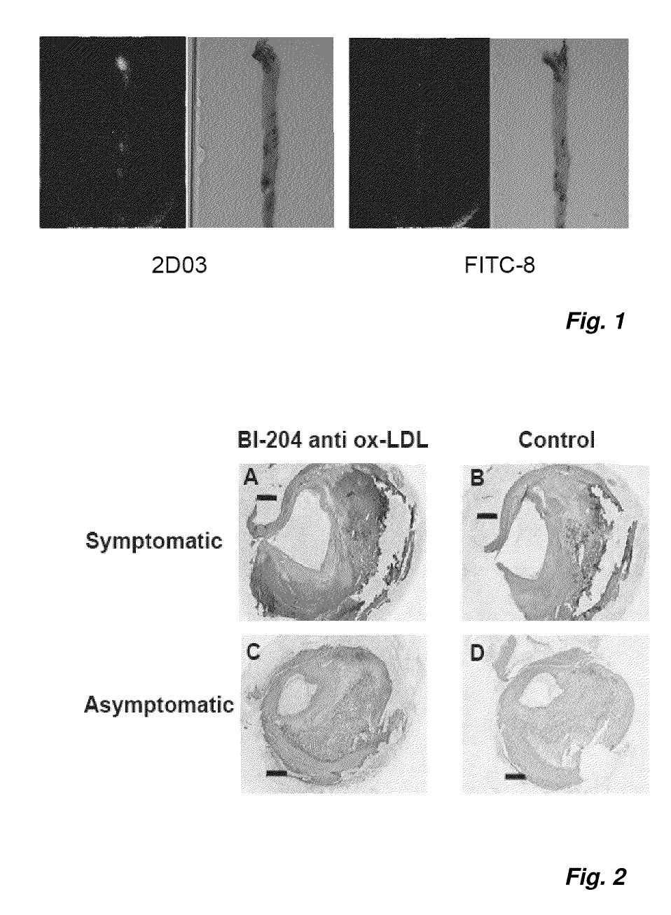

It has subsequently been demonstrated that when injected into atherosclerotic animals this antibody localizes specifically to atherosclerotic plaques (FIG. 1).

Using a panel of unstable (i.e. plaques giving rise to clinical events such as stroke) and stable (i.e. clinically silent plaques) human carotid atherosclerotic plaques we have also demonstrated that these antibodies preferentially target unstable plaque (FIG. 2).

### SUMMARY OF THE PRESENT INVENTION

The present invention aims at specifically target human unstable atherosclerotic plaque using certain fusion protein between oxidized specific antibody fusion and conjugated proteins.

### DETAILED DESCRIPTION OF THE DRAWINGS

FIG. 1 illustrates localization of radio labeled 2D03 anti-ox LDL and the control (FITC-8) antibodies to atherosclerotic plaques in hyper-cholesterolemic mice. Red color in the right panels depicts lipids in atherosclerotic in the aorta of the animals.

FIG. 2 illustrates immunohistochemical localization (brown color) of the BI-204 ox-LDL antibody and an unspecific control antibody to unstable (clinically symptomatic) and stable (clinically asymptomatic) human atherosclerotic plaques.

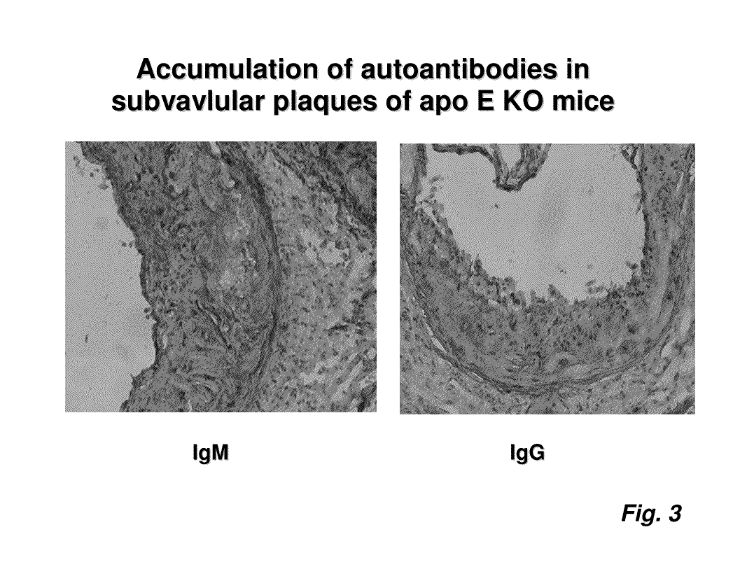

FIG. 3 show accumulation of autoantibodies in atherosclerotic plaques of hypercholesterolemic mice demonstrating that atherosclerosis involves autoimmunity against structures present in the plaques.

### DETAILED DESCRIPTION OF THE PRESENT INVENTION

The present invention in particular relates to complete oxidized LDL specific IgG fused or conjugated with at least one tissue stabilizing factor to be used in a medicine.

In particular the present invention relates to complete oxidized LDL specific IgG fused or conjugated with at least one of the proteins of the group IL-10, TIMPs, and TGFβs,

In particular the present invention relates to complete oxidized LDL specific IgG combined with IL-10 as fusion protein in medicine, in particular for treatment of atherosclerosis and prevention of clinical events in patients with atherosclerosis.

A preferred embodiment of the invention relates to complete oxidized LDL specific IgG combined with TGFβ as fusion protein in medicine, in particular for treatment of atherosclerosis and prevention of clinical events in patients with atherosclerosis

A preferred embodiment of the invention relates to complete oxidized LDL specific IgG combined with TIMP as fusion protein in medicine, in particular for treatment of atherosclerosis and prevention of clinical events in patients with atherosclerosis

A preferred embodiment of the invention relates to complete oxidized LDL specific IgG combined with IL-10 as conjugated protein in medicine, in particular for treatment of atherosclerosis and prevention of clinical events in patients with atherosclerosis

A preferred embodiment of the invention relates to complete oxidized LDL specific IgG combined with TGFβ as conjugated protein in medicine, in particular for treatment of atherosclerosis and prevention of clinical events in patients with atherosclerosis

A preferred embodiment of the invention relates to complete oxidized LDL specific IgG combined with TIMP as conjugated protein in medicine, in particular for treatment of atherosclerosis and prevention of clinical events in patients with atherosclerosis

A preferred embodiment of the invention relates to complete oxidized

LDL specific single chains combined with IL-10 as fusion protein in medicine, in particular for treatment of atherosclerosis and prevention of clinical events in patients with atherosclerosis.

A further preferred embodiment of the invention relates to complete oxidized LDL specific Fab fragments combined with IL-10 as fusion protein in medicine, f in particular or treatment of atherosclerosis and prevention of clinical events in patients with atherosclerosis.

A still further preferred embodiment of the invention relates to complete oxidized LDL specific IgG raised against the peptides derived from apoB-100 protein

| | |
|---|---|
| FLDTVYGNCSTHFTVKTRKG | (SEQ. ID. NO. 1) |
| PQCSTHILQWLKRVHANPLL | (SEQ. ID. NO. 2) |
| VISIPRLQAEARSEILAHWS | (SEQ. ID. NO. 3) |
| IALDDAKINFNEKLSQLQTY | (SEQ. ID. NO. 4) |
| KTTKQSFDLSVKAQYKKNKH | (SEQ. ID. NO. 5) |
| EEEMLENVSLVCPKDATRFK | (SEQ. ID. NO. 6) |
| GSTSHHLVSRKSISAALEHK | (SEQ. ID. NO. 7) |
| IENIDFNKSGSSTASWIQNV | (SEQ. ID. NO. 8) |
| IREVTQRLNGEIQALELPQK | (SEQ. ID. NO. 9) |
| EVDVLTKYSQPEDSLIPFFE | (SEQ. ID. NO. 10) |
| ALLVPPETEEAKQVLFLDTV | (SEQ. ID. NO. 11) |
| IEIGLEGKGFEPTLEALFGK | (SEQ. ID. NO. 12) |
| SGASMKLTTNGRFREHNAKF | (SEQ. ID. NO. 13) |
| NLIGDFEVAEKINAFRAKVH | (SEQ. ID. NO. 14) |
| GHSVLTAKGMALFGEGKAEF | (SEQ. ID. NO. 15) |
| FKSSVITLNTNAELFNQSDI | (SEQ. ID. NO. 16) |
| FPDLGQEVALNANTKNQKIR | (SEQ. ID. NO. 17) |

A further aspect of the invention relates to antibodies raised against apoB-100 protein fragments as given above, which antibodies have a variable heavy region (V_{H}) selected from the group of nucleic acid sequences consisting of:

(SEQ. ID. NO. 101)
GAGGTGCAGCTGTTGGAGTCTGGGGGAGGCTTGGTACAGCCTGGGGGGTC
CCTGAGACTCTCCTGTGCAGCCTCTGGATTCACCTTCAATAACGCCTGGA
TGAGCTGGGTCCGCCAGGCTCCAGGGAAGGGGCTGGAGTGGGTCTCATCC
ATTAGTAGTAGTAGTAGTTACATATACTACGCAGACTCAGTGAAGGGCCG
ATTCACCATCTCCAGAGACAATTCCAAGAACACGCTGTATCTGCAAATGA
ACAGCCTGAGAGCCGAGGACACTGCCGTGTATTACTGTGCGAGAGTCAGT
AGGTACTACTACGGACCATCTTTCTACTTTGACTCCTGGGGCCAGGGTAC
ACTGGTCACCGTGAGCAGC
(SEQ. ID. NO. 103)
GAGGTGCAGCTGTTGGAGTCTGGGGGAGGCTTGGTACAGCCTGGGGGGTC
CCTGAGACTCTCCTGTGCGGCCTCTGGATTCACCTTCAGTGACTACTACA
TGAGCTGGGTCCGCCAGGCTCCCGGGAAGGGGCTGGAGTGGGTATCGGGT
GTTAGTTGGAATGGCAGTAGGACGCACTATGCAGACTCTGTGAAGGGCCG
ATTCACCATCTCCAGAGACAATTCCAAGAACACGCTGTATCTGCAAATGA
ACAGCCTGAGAGCCGAGGACACTGCCGTGTATTACTGTGCGAGAGCGGCT
AGGTACTCCTACTACTACTACGGTATGGACGTCTGGGGCCAAGGTACACT
GGTCACCGTGAGCAGC
(SEQ. ID. NO. 105)
GAGGTGCAGCTGTTGGAGTCTGGGGGAGGCTTGGTACAGCCTGGGGGGTC
CCTGAGACTCTCCTGTGCAGCCTCTGGATTCACCTTTAGTAGCTATTGGA
TGAGCTGGGTCCGCCAGGCTCCAGGGAAGGGGCTGGAGTGGGTCTCAAGT
ATCAGTGGTAGTGGTCGTAGGACATACTACGCAGACTCCGTGCAGGGCCG
GTTCACCATCTCCAGAGACAATTCCAAGAACACGCTGTATCTGCAAATGA
ACAGCCTGAGAGCCGAGGACACTGCCGTGTATTACTGTGCGAGATTGGTC
TCCTATGGTTCGGGGAGTTTCGGTTTTGACTACTGGGGCCAAGGTACACT
GGTCACCGTGAGCAGC
(SEQ. ID. NO. 107)
GAGGTGCAGCTGTTGGAGTCTGGGGGAGGCTTGGTACAGCCTGGGGGGTC
CCTGAGACTCTCCTGTGCAGCCTCTGGATTCACCTTCAGTAACGCCTGGA
TGAGCTGGGTCCGCCAGGTTCCAGGGAAGGGGCTGGAGTGGGTCTCAACT
CTTGGTGGTAGTGGTGGTGGTAGCACATACTACGCAGACTCCGTGAAGGG
CCGGTTCACCATCTCCAGAGACAATTCCAAGAACACGCTGTATCTGCAAA
TGAACAGCCTGAGAGCCGAGGACACTGCCGTGTATTACTGTGCGAAGTTA
GGGGGGCGATCCCGATATGGGCGGTGGCCCCGCCAATTTGACTACTGGGG
CCAAGGTACACTGGTCACCGTGAGCAGC
(SEQ. ID. NO. 109)
GAGGTGCAGCTGTTGGAGTCTGGGGGAGGCTTGGTACAGCCTGGGGGGTC
CCTGAGACTCTCCTGTGCAGCCTCTGGATTCACCTTCAGTGACTACTACA
TGAGCTGGATCCGCCAGGCTCCAGGGAAGGGGCTGGAGTGGGTCTCAAGT
ATCAGTGGCCGTGGGGGTAGTTCCTACTACGCAGACTCCGTGAGGGGCCG
GTTCACCATCTCCAGAGACAATTCCAAGAACACGCTGTATCTGCAAATGA
ACAGCCTGAGAGCCGAGGACACTGCCGTGTATTACTGTGCGAGACTTTCC
TACAGCTATGGTTACGAGGGGGCCTACTACTTTGACTACTGGGGCCAGGG
TACACTGGTCACCGTGAGCAGC
(SEQ. ID. NO. 111)
GAGGTGCAGCTGTTGGAGTCTGGGGGAGGCTTGGTACAGCCTGGGGGGTC
CCTGAGACTCTCCTGTGCAGCCTCTGGATTCACCTTTAGCAGCTATGCCA
TGAGCTGGGTCCGCCAGGCTCCAGGGAAGGGGCTGGAGTGGGTCTCATCC
ATTAGTAGTAGTGGTCGTTTCATTTACTACGCAGACTCAATGAAGGGCCG
CTTCACCATCTCCAGAGACAATTCCAAGAACACGCTGTATCTGCAAATGA
ACAGCCTGAGAGCCGAGGACACTGCCGTGTATTACTGTACGAGGCTCCGG
AGAGGGAGCTACTTCTGGGCTTTTGATATCTGGGGCCAAGGTACACTGGT
CACCGTGAGCAGC
(SEQ. ID. NO. 113)
GAGGTGCAGCTGTTGGAGTCTGGGGGAGGCTTGGTACAGCCTGGGGGGTC
CCTGAGACTCTCCTGTGCAGCCTCTGGATTCACCTTTAGAACGTATTGGA
TGACCTGGGTCCGCCAGGCTCCAGGGAAGGGGCTGGAGTGGGTCTCATCT
ATTAGCAGTAGCAGTAATTACATATTCTACGCAGACTCAGTGAAGGGCCG
ATTCACCATCTCCAGAGACAATTCCAAGAACACGCTGTATCTGCAAATGA
ACAGCCTGAGAGCCGAGGACACTGCCGTGTATTACTGTGCGAGACTCAGA
CGGAGCAGCTGGTACGGGGGGTACTGGTTCGACCCCTGGGGCCAAGGTAC
ACTGGTCACCGTGAGCTCA
(SEQ. ID. NO. 115)
GAGGTGCAGCTGTTGGAGTCTGGGGGAGGCTTGGTACAGCCTGGGGGGTC
CCTGAGACTCTCCTGTGCAGCCTCTGGATTCACCTTCAGTAGCAACTACA
TGAGCTGGGTCCGCCAGGCTCCAGGGAAGGGGCTGGAGTGGGTCTCATCC
ATTAGTAGTAGTAGTAGTTACATATACTACGCAGACTCAGTGAAGGGCCG
ATTCACCATCTCCAGAGACAATTCCAAGAACACGCTGTATCTGCAAATGA
ACAGCCTGAGAGCCGAGGACACTGCCGTGTATTACTGTGCGAGAGTAGGC
CGGTATAACTGGAAGACGGGGCATGCTTTTGATATCTGGGGCCAGGGTAC
ACTGGTCACCGTGAGCTCA
(SEQ. ID. NO. 117)
GAGGTGCAGCTGTTGGAGTCTGGGGGAGGCTTGGTACAGCCTGGGGGGTC
CCTGAGACTCTCCTGTGCAGCCTCTGGATTCACCTTCCGTGACTACTACG
TGAGCTGGATCCGCCAGGCTCCAGGGAAGGGGCTGGAGTGGGTCTCAAGT
ATTAGTGGTAGTGGGGGTAGGACATACTACGCAGACTCCGTGGAGGGCCG
GTTCACCATCTCCAGAGACAATTCCAAGAACACGCTGTATCTGCAAATGA
ACAGCCTGAGAGCCGAGGACACTGCCATGTATTACTGTGCCAGAGTATCC
GCCCTTCGGAGACCCATGACTACAGTAACTACTTACTGGTTCGACCCCTG
GGGCCAAGGTACACTGGTCACCGTGAGCTCA
(SEQ. ID. NO. 119)
GAGGTGCAGCTGTTGGAGTCTGGGGGAGGCTTGGTACAGCCTGGGGGGTC
CCTGAGACTCTCCTGTGCAGCCTCTGGATTCACCTTCAGTAACGCCTGGA
TGAGCTGGGTCCGCCAGGCTCCAGGGAAGGGGCTGGAGTGGGTCTCCGCT
ATTAGTGGTAGTGGTAACACATACTATGCAGACTCCGTGAAGGGCCGGTT
CACCATCTCCAGAGACAATTCCAAGAACACGCTGTATCTGCAAATGAACA
GCCTGAGAGCCGAGGACACTGCCGTGTATTACTGTGCGAGAGCCTCCCAC
CGTATATTAGGTTATGCTTTTGATATCTGGGGCCAGGGTACACTGGTCAC
CGTGAGCTCA
(SEQ. ID. NO. 121)
GAGGTGCAGCTGTTGGAGTCTGGGGGAGGCTTGGTACAGCCTGGGGGGTC
CCTGAGACTCTCCTGTGCAGCCTCTGGATTCACCTTCAGTAACGCCTGGA
TGAGCTGGGTCCGCCAGGCTCCAGGGAAGGGGCTGGAGTGGGTCTCAAGT
ATTAGTGTTGGTGGACATAGGACATATTATGCAGATTCCGTGAAGGGCCG
GTCCACCATCTCCAGAGACAATTCCAAGAACACGCTGTATCTGCAAATGA
ACAGCCTGAGAGCCGAGGACACTGCCGTGTATTACTGTGCACGGATACGG
GTGGGTCCGTCCGGCGGGGCCTTTGACTACTGGGGCCAGGGTACACTGGT
CACCGTGAGCTCA
(SEQ. ID. NO. 123)
GAGGTGCAGCTGTTGGAGTCTGGGGGAGGCTTGGTACAGCCTGGGGGGTC
CCTGAGACTCTCCTGTGCAGCCTCTGGATTCACCTTCAGTAACGCCTGGA
TGAGCTGGGTCCGCCAGGCTCCAGGGAAGGGGCTGGAGTGGGTCTCATCC
ATTAGTAGTAGTAGTAGTTACATATACTACGCAGACTCAGTGAAGGGCCG
ATCCACCATCTCCAGAGACAATTCCAAGAACACGCTGTATCTGCAAATGA
ACAGCCTGAGAGCCGAGGACACTGCCGTGTATTACTGTGCGAGGCTCACA
AATATTTTGACTGGTTATTATACCTCAGGATATGCTTTTGATATCTGGGG
CCAAGGTACACTGGTCACCGTGAGCTCA
(SEQ. ID. NO. 125)
GAGGTGCAGCTGTTGGAGTCTGGGGGAGGCTTGGTACAGCCTGGGGGGTC
CCTGAGACTCTCCTGTGCAGCCTCTGGATTCACCTTCAGTAGTTCTTGGA
TGAGTTGGGTCCGCCAGGCTCCAGGGAAGGGGCTGGAGTGGGTCTCATCC
ATTAGTAGTAGTAGTAGTTACATATACTACGCAGACTCAGTGAAGGGCCG
ATTCACCATCTCCAGAGACAATTCCAAGAACACGCTGTATCTGCAAATGA
ACAGCCTGAGAGCCGAGGACACTGCCGTGTATTACTGTGCGAGAGTAGGG
AACTACGGTTTCTACCACTACATGGACGTCTGGGGCCAAGGTACACTGGT
CACCGTGAGCTCA
(SEQ. ID. NO. 127)
GAGGTGCAGCTGTTGGAGTCTGGGGGAGGCTTGGTACAGCCTGGGGGGTC
CCTGAGACTCTCCTGTGCAGCCTCTGGATTCACCTTTAGTAGCTATTGGA
TGAGCTGGGTCCGCCAGGCTCCAGGGAAGGGGCTGGAGTGGGTCTCATCC
ATTAGTAGTAGTAGTAGTTACATATACTACGCAGACTCAGTGAAGGGCCG
ATTCACCATCTCCAGAGACAATTCCAAGAACACGCTGTATCTGCAAATGA
ACAGCCTGAGAGCCGAGGACACTGCCGTGTATTACTGTGCGAGAATTAAA
CGGTTACGATTCGGCTGGACCCCTTTTGACTACTGGGGCCAGGGTACACT
GGTCACCGTGAGCTCA
(SEQ. ID. NO. 129)
TCCTGTGCAGCCTCTGGATTCACCTTCAGTAACGCCTGGATGAGCTGGGT
CCGCCAGGCTCCAGGGAAGGGGCTGGAGTGGGTCTCATCCATTAGTAGTA
GTAGTAGTTACATATACTACGCAGACTCAGTGAAGGGCCGATTCACCATC
TCCAGAGACAATTCCAAGAACACGCTGTATCTGCAAATGAACAGCCTGAG
AGCCGAGGACACTGCCGTGTATTACTGTGCGAGAGTCAATAGCAAAAAGT
GGTATGAGGGCTACTTCTTTGACTACTGGGGCCAGGGTACACTGGTCACC
GTGAGCTCA
(SEQ. ID. NO. 131)
GAGGTGCAGCTGTTGGAGTCTGGGGGAGGCTTGGTACAGCCTGGGGGGTC
CCTGAGACTCTCCTGTGCAGCCTCTGGATTCACCTTCAGTAACGCCTGGA
TGAGCTGGGTCCGCCAGGCTCCAGGGAAGGGGCTGGAGTGGGTCTCATCC
ATTAGTACTAGTAGTAATTACATATACTACGCAGACTCAGTGAAGGGCCG
GTTCACCATCTCCAGAGACAATTCCAAGAACACGCTGTATCTGCAAATGA
ACAGCCTGAGAGCCGAGGACACTGCCGTGTATTACTGTGCGAGAGTCAAG
AAGTATAGCAGTGGCTGGTACTCGAATTATGCTTTTGATATCTGGGGCCA
AGGTACACTGGTCACCGTGAGCTCA.

A further aspect of the invention relates to antibodies against apoB-100 protein fragments as given above, which antibodies have a variable light region (V_{L}) selected from the group of nucleic acid sequences consisting of:

(SEQ. ID. NO. 102)
CAGTCTGTGCTGACTCAGCCACCCTCAGCGTCTGGGACCCCCGGGCAGAG
GGTCACCATCTCCTGCTCTGGAAGCAGGTCCAACATTGGGAATAATTATG
TATCCTGGTATCAGCAGCTCCCAGGAACGGCCCCCAAACTCCTCATCTAT
GGTAACAACAATCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGCATGGGATGACAGCCTGAATGGTCATTGG
GTGTTCGGCGGAGGAACCAAGCTGACGGTCCTAGGT
(SEQ. ID. NO. 104)
CAGTCTGTGCTGACTCAGCCACCCTCAGCGTCTGGGACCCCCGGGCAGAG
GGTCACCATCTCCTGTTCTGGAAGCAGCTCCAACATCGGAAATAATGCTG
TAAACTGGTATCAGCAGCTCCCAGGAACGGCCCCCAAACTCCTCATCTAT
GGGAATGATCGGCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTCAGACCTGGGGCACTGGCCGGGGGGTATTCGGC
GGAGGAACCAAGCTGACGGTCCTAGGT
(SEQ. ID. NO. 106)
CAGTCTGTGCTGACTCAGCCACCCTCAGCGTCTGGGACCCCCGGGCAGAG
GGTCACCATCTCTTGTTCTGGAAGCAGCTCCAATATCGGAAGTAATTATG
TATCCTGGTATCAGCAGCTCCCAGGAACGGCCCCCAAACTCCTCATCTAT
GGTAACTACAATCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGCATGGGATGACAGCCTGAGTGGTTGGGTG
TTCGGCGGAGGAACCAAGCTGACGGTCCTAGGT
(SEQ. ID. NO. 108)
CAGTCTGTGCTGACTCAGCCACCCTCAGCGTCTGGGACCCCCGGGCAGAG
GGTCACCATCTCCTGCTCTGGAAGCAGCTCCAACATTGGAAATAACTATG
TATCCTGGTATCAGCAGCTCCCAGGAACGGCCCCCAAACTCCTCATCTAT
AGTAATAATCAGCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGCATGGGATGACAGCCTGAGTCATTGGCTG
TTCGGCGGAGGAACCAAGCTGACGGTCCTAGGT
(SEQ. ID. NO. 110)
CAGTCTGTGCTGACTCAGCCACCCTCAGCGTCTGGGACCCCCGGGCAGAG
GGTCACCATCTCCTGCTCTGGAAGCAGCTCCAACATTGGGAATAATTATG
TATCCTGGTATCAGCAGCTCCCAGGAACGGCCCCCAAACTCCTCATCTAT
AGGAATAATCAGCGGCCCTCAGGGGTCCCTGACCGATTCTCTGGCTCCAA
GTCTGGCACCTTAGCCTCCCTGGCCATCAGTGGGCTCCGGTCCGAGGATG
AGGCTGATTATTACTGTGCAACCTGGGATGACAGCCTGAATGGTTGGGTG
TTCGGCGGAGGAACCAAGCTGACGGTCCTAGGT
(SEQ. ID. NO. 112)
CAGTCTGTGCTGACTCAGCCACCCTCAGCGTCTGGGACCCCCGGGCAGAG
GGTCACCATCTCCTGTTCTGGAAGCAGCTCCAACATTGGCGGTGAGTCTG
TATCCTGGTATCAGCAGCTCCCAGGAACGGCCCCCAAACTCCTCATCTAT
AGTAATAATCAGCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGCATGGGATGACAGCCTGAATGGTTGGGTG
TTCGGCGGAGGAACCAAGCTGACGGTCCTAGGT
(SEQ. ID. NO. 114)
CAGTCTGTGCTGACTCAGCCACCCTCAGCGTCTGGGACCCCCGGGCAGAG
GGTCACCATCTCCTGCTCTGGAAGCAGCTCCAACATTGGGAATAATTATG
TATCCTGGTATCAGCAGCTCCCAGGAACGGCCCCCAAACTCCTCATCTAT
AGGAATAATCAGCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGCATGGGATGACAGCCTGAATGGTATTGGG
TGTTCGGCGGAGGAACCAAGCTGACGGTCCTAGGT
(SEQ. ID. NO. 116)
CAGTCTGTGCTGACTCAGCCACCCTCAGCGTCTGGGACCCCCGGGCAGAG
GGTCACCATCTCCTGCTCTGGAAGGACCTACAACATTGGAAATAATTATG
TATCGTGGTATCAGCAGCTCCCAGGAACGGCCCCCAAACTCCTCATCTAT
GGTAACATCAATCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGCATGGGATGTCAGGCTGAATGGTTGGGTG
TTCGGCGGAGGAACCAAGCTGACGGTCCTAGGT
(SEQ. ID. NO. 118)
CAGTCTGTGCTGACTCAGCCACCCTCAGCGTCTGGGACCCCCGGGCAGAG
GGTCACCATCTCCTGCTCTGGAAGGAGCTCCAACATTGGGAATAGTTATG
TCTCCTGGTATCAGCAGCTCCCAGGAACGGCCCCCAAACTCCTCATCTAT
AGGAATAATCAGCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGGATGGGATGACACCCTGCGTGCTTGGGTG
TTCGGCGGAGGAACCAAGCTGACGGTCCTAGGT
(SEQ. ID. NO. 120)
CAGTCTGTGCTGACTCAGCCACCCTCAGCGTCTGGGACCCCCGGGCAGAG
GGTCACCATCTCTTGTTCTGGAAGCCGCTCCAACATCGGGAGAAATGCTG
TTAGTTGGTATCAGCAGCTCCCAGGAACGGCCCCCAAACTCCTCATCTAT
GCTAACAGCAATCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGCATGGGATGGCAGCCTGAATGGTTGGGTG
TTCGGCGGAGGAACCAAGCTGACGGTCC
(SEQ. ID. NO. 122)
CAGTCTGTGCTGACTCAGCCACCCTCAGCGTCTGGGACCCCCGGGCAGAG
GGTCACCATCTCCTGCTCTGGAAGCAACACCAACATTGGGAAGAACTATG
TATCTTGGTATCAGCAGCTCCCAGGAACGGCCCCCAAACTCCTCATCTAT
GCTAATAGCAATCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCGTCATGGGATGCCAGCCTGAATGGTTGGGTA
TTCGGCGGAGGAACCAAGCTGACGGTCCTAGGT
(SEQ. ID. NO. 124)
CAGTCTGTGCTGACTCAGCCACCCTCAGCGTCTGGGACCCCCGGGCAGAG
GGTCACCATCTCCTGCTCTGGAAGCACCTCCAACATTGGGAAGAATTATG
TATCCTGGTATCAGCAGCTCCCAGGAACGGCCCCCAAACTCCTCATCTAT
GGTAACAGCAATCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGCATGGGATGCCAGCCTCAGTGGTTGGGTG
TTCGGCGGAGGAACCAAGCTGACGGTCCTAGGT
(SEQ. ID. NO. 126)
CAGTCTGTGCTGACTCAGCCACCCTCAGCGTCTGGGACCCCCGGGCAGAG
GGTCACCATCTCTTGTTCTGGAGGCAGCTCAAACATCGGAAAAAGAGGTG
TAAATTGGTATCAGCAGCTCCCAGGAACGGCCCCCAAACTCCTCATCTAT
GGTAACAGAAATCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCTACATGGGATTACAGCCTCAATGCTTGGGTG
TTCGGCGGAGGAACCAAGCTGACGGTCCTAGGT
(SEQ. ID. NO. 128)
CAGTCTGTTCTGACTCAGCCACCCTCAGCGTCTGGGACCCCCGGGCAGAG
GGTCACCATCTCCTGTTCTGGAAGCAGCTCCAACATCGGAAATAATGGTG
TAAACTGGTATCAGCAGCTCCCAGGAACGGCCCCCAAACTCCTCATCTAT
GGTAACAACAATCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGCATGGGATGACAGCCTGCGTGGTTGGCTG
TTCGGCGGAGGAACCAAGCTGACGGTCCTAGGT
(SEQ. ID. NO. 130)
CAGTCTGTGCTGACTCAGCCACCCTCAGCGTCTGGGACCCCCGGGCAGAG
GGTCACCATCTCCTGCTCTGGAAGCAGCTCCAACATTGGGAATAATTATG
TATCCTGGTATCAGCAGCTCCCAGGAACGGCCCCCAAACTCCTCATCTAT
GGTAACAGCAATCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGCATGGGATGACAGTCTGAGTGGTTGGGTG
TTCGGCGGAGGAACCAAGCTGACGGTCCTAGGT
(SEQ. ID. NO. 132)
CAGTCTGTGCTGACTCAGCCACCCTCAGCGTCTGGGACCCCCGGGCAGAG
GGTCACCATCTCCTGCTCTGGAAGCAGCTCCAGCATTGGGAATAATTTTG
TATCCTGGTATCAGCAGCTCCCAGGAACGGCCCCCAAACTCCTCATCTAT
GACAATAATAAGCGACCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTACTGTGCAGCATGGGATGACAGCCTGAATGGTTGGGTG
TTCGGCGGAGGAACCAAGCTGACGGTCCTAGGT

A further aspect of the present invention relates to the particular fusion and conjugated proteins mentioned above thus including complete oxidized LDL specific IgG fused or conjugated with at least one tissue stabilizing factor to be used in a medicine.

A preferred embodiment of the invention relates to complete oxidized LDL specific IgG fused or conjugated with at least one of the proteins of the group IL-10, TIMPs, and TGFβs,

A preferred embodiment of the invention relates to complete oxidized LDL specific IgG combined with IL-10 as fusion protein in medicine, in particular for treatment of atherosclerosis and prevention of clinical events in patients with atherosclerosis.

A preferred embodiment of the invention relates to complete oxidized LDL specific IgG combined with TGFβ as fusion protein in medicine, in particular for treatment of atherosclerosis and prevention of clinical events in patients with atherosclerosis.

A preferred embodiment of the invention relates to complete oxidized LDL specific IgG combined with TIMP as fusion protein in medicine, in particular for treatment of atherosclerosis and prevention of clinical events in patients with atherosclerosis

A preferred embodiment of the invention relates to complete oxidized LDL specific IgG combined with IL-10 as conjugated protein in medicine, in particular for treatment of atherosclerosis and prevention of clinical events in patients with atherosclerosis

A preferred embodiment of the invention relates to complete oxidized LDL specific IgG combined with TGFα as conjugated protein in medicine, in particular for treatment of atherosclerosis and prevention of clinical events in patients with atherosclerosis.

A preferred embodiment of the invention relates to complete oxidized LDL specific IgG combined with TIMP as conjugated protein in medicine, in particular for treatment of atherosclerosis and prevention of clinical events in patients with atherosclerosis

A preferred embodiment of the invention relates to complete oxidized LDL specific single chains combined with IL-10 as fusion protein in medicine, in particular for treatment of atherosclerosis and prevention of clinical events in patients with atherosclerosis.

A further preferred embodiment of the invention relates to complete oxidized LDL specific Fab fragments combined with IL-10 as fusion protein in medicine, f in particular or treatment of atherosclerosis and prevention of clinical events in patients with atherosclerosis.

A further preferred embodiment of the invention relates to complete oxidized LDL specific single chains or Fab fragments raised against one or more of the apoB-100 peptides with SEQ. ID. NO. 1, SEQ. ID. NO. 2, SEQ. ID. NO. 3, SEQ. ID. NO. 4, SEQ. ID. NO. 5, SEQ. ID. NO. 6, SEQ. ID. NO. 7, SEQ. ID. NO. 8, SEQ. ID. NO. 9, SEQ. ID. NO. 10, SEQ. ID. NO. 11, SEQ. ID. NO. 12, SEQ. ID. NO. 13, SEQ. ID. NO. 14, SEQ. ID. NO. 15, SEQ. ID. NO. 16, and SEQ. ID. NO. 17 combined with IL-10 as fusion protein in medicine, in particular for treatment of atherosclerosis and prevention of clinical events in patients with atherosclerosis.

A still further aspect of the invention relates to a pharmacetical composition comprising complete oxidized LDL specific IgG fused or conjugated with at least one tissue stabilizing factor to be used in a medicine in combination with suitable adjuvants and excipients.

A further preferred embodiment of the invention relates to a pharmaceutical composition comprising complete oxidized LDL specific IgG fused or conjugated with at least one of the proteins of the group IL-10, TIMPs, and TGFβs to be used in a medicine

A further preferred embodiment of the invention relates to a pharmaceutical composition comprising complete oxidized LDL specific IgG combined with IL-10 as fusion protein to be used in a medicine for treatment of atherosclerosis and prevention of clinical events in patients with atherosclerosis.

A further preferred embodiment of the invention relates to a pharmaceutical composition comprising complete oxidized LDL specific IgG combined with TGFβ as fusion protein to be used in a medicine for treatment of atherosclerosis and prevention of clinical events in patients with atherosclerosis.

A further preferred embodiment of the invention relates to a pharmaceutical composition comprising complete oxidized LDL specific IgG according to claim 1 combined with TIMP as fusion protein to be used in a medicine for treatment of atherosclerosis and prevention of clinical events in patients with atherosclerosis.

A further preferred embodiment of the invention relates to a pharmaceutical composition comprising complete oxidized LDL specific IgG combined with IL-10 as conjugated protein to be used in a medicine for treatment of atherosclerosis and prevention of clinical events in patients with atherosclerosis.

A further preferred embodiment of the invention relates to a pharmaceutical composition comprising complete oxidized LDL specific IgG combined with TGFβ as conjugated protein to be used in a medicine for treatment of atherosclerosis and prevention of clinical events in patients with atherosclerosis.

A further preferred embodiment of the invention relates to a pharmaceutical composition comprising complete oxidized LDL specific IgG combined with TIMP as conjugated protein to be used in a medicine for treatment of atherosclerosis and prevention of clinical events in patients with atherosclerosis.

A further preferred embodiment of the invention relates to a pharmaceutical composition comprising complete oxidized LDL specific IgG single chains combined with IL-10 as fusion protein to be used in a medicine for treatment of atherosclerosis and prevention of clinical events in patients with atherosclerosis.

A further preferred embodiment of the invention relates to a pharmaceutical composition comprising complete oxidized LDL specific IgG Fab fragments combined with IL-10 as fusion protein to be used in a medicine for treatment of atherosclerosis and prevention of clinical events in patients with atherosclerosis.

A further preferred embodiment of the invention relates to a pharmaceutical composition comprising complete oxidized LDL specific IgG single chains or Fab fragments raised against one or more apoB-100 peptides combined with IL-10 as fusion protein for treatment of atherosclerosis and prevention of clinical events in patients with atherosclerosis, which peptides are those with SEQ. ID. NO. 1, SEQ. ID. NO. 2, SEQ. ID. NO. 3, SEQ. ID. NO. 4, SEQ. ID. NO. 5, SEQ. ID. NO. 6, SEQ. ID. NO. 7, SEQ. ID. NO. 8, SEQ. ID. NO. 9, SEQ. ID. NO. 10, SEQ. ID. NO. 11, SEQ. ID. NO. 12, SEQ. ID. NO. 13, SEQ. ID. NO. 14, SEQ. ID. NO. 15, SEQ. ID. NO. 16 and/or SEQ. ID. NO. 17.

A still further aspect of the invention relates to a method for treating atherosclerosis and prevention of clinical events in patients with atherosclerosis wherein a therapeutically effective amount of a complete oxidized LDL specific IgG fused or conjugated with at least one tissue stabilizing factor is administered to a patient suffering from atherosclerosis.

Taken together these observations demonstrate the possibility to specifically target human unstable atherosclerotic plaques using complete, single chain or Fab fragments of oxidized LDL specific antibodies. By constructing recombinant fusion proteins or conjugates of complete, single chain or Fab fragments of oxidized LDL specific antibodies with factors that can act locally to stabilize plaques such as IL-10, TGFβ and it will be possible to develop local plaque stabilizing therapy. Accordingly, by using this type of oxidized LDL-specific antibody fusion or conjugate constructs it would become possible to coat oxidized LDL in plaques with factors that effectively counteracts the plaque destabilization effects of oxidized LDL. It is anticipated that this type of therapy will be more effective and associated with fewer side effects that existing systemic therapies.

The figures of FIG. 3 show accumulation of autoantibodies in atherosclerotic plaques of hypercholesterolemic mice demonstrating that atherosclerosis involves autoimmunity against structures present in the plaques.

Administration of the protein is normally carried out by injection, such as subcutaneous injection, intravenous injection, intramuscular injection or intraperitoneal injection. A first immunizing dosage can be 0.001 to 400 mg per patient depending on body weight, age, and other physical and medical conditions. In particular situations a local administration of a solution containing the protein via catheter to the coronary vessels is possible as well. Oral preparations may be contemplated as well, although particular precautions must be taken to admit absorption into the blood stream. Further nasal inhalation formulations may be contemplated, as well. An injection dosage may contain 0.5 to 99.5% by weight of the protein of the present invention.

The protein is normally administered as such or may be linked to cationized bovine serum albumin, and using aluminium hydroxide or Freund's complete and incomplete adjuvants as an adjuvant. Other adjuvants known in the art can be used as well.

The protein can also be used as therapeutic agent in patients already suffering from an atherosclerosis. Thus any suitable administration route can be used for adding the protein of the invention.

### REFERENCES

1. Hansson G K: Inflammation, atherosclerosis, and coronary artery disease. N Engl J Med 2005; 352(16): 1685-95.
2. Glass C K, Witztum J L: Atherosclerosis: The Road Ahead. Cell 2001; 104: 503-516.
3. Binder C J, Shaw P X, Chang M K, et al.: The role of natural antibodies in atherogenesis. J Lipid Res 2005; 46(7): 1353-63.
4. Binder C J, Chang M K, Shaw P X, et al.: Innate and acquired immunity in atherogenesis. Nat Med 2002; 8(11): 1218-26.
5. Nilsson J, Hansson G K: Autoimmunity in atherosclerosis: a protective response losing control? J Intern Med 2008; 263(5): 464-78.
6. Palinski W, Miller E, Witztum J L: Immunization of low density lipoprotein (LDL) receptor-deficient rabbits with homologous malondialdehyde-modified LDL reduces atherogenesis. Proc Natl Acad Sci USA 1995; 92(3): 821-5.
7. Ameli S, Hultgardh-Nilsson A, Regnstrom J, et al.: Effect of immunization with homologous LDL and oxidized LDL on early atherosclerosis in hypercholesterolemic rabbits. Arterioscler Thromb Vasc Biol 1996; 16(8): 1074-9.
8. Zhou X, Caligiuri G, Hamsten A, Lefvert A K, Hansson G K: LDL immunization induces T-cell-dependent antibody formation and protection against atherosclerosis. Arterioscler Thromb Vasc Biol 2001; 21(1): 108-14.
9. Nilsson J, Hansson G K, Shah P K: Immunomodulation of atherosclerosis: implications for vaccine development. Arterioscler Thromb Vasc Biol 2005; 25(1): 18-28.
10. Fredrikson G N, Hedblad B, Berglund G, et al.: Identification of immune responses against aldehyde-modified peptide sequences in apo B-100 associated with cardiovascular disease. Arterioscler Thromb Vasc Biol 2003; 23(5): 872-8.
11. Fredrikson G N, Soderberg I, Lindholm M, et al.: Inhibition of Atherosclerosis in ApoE-Null Mice by Immunization with ApoB-100 Peptide Sequences. Arterioscler Thromb Vasc Biol 2003; 23(5): 879-84.
12. Chyu K Y, Zhao X, Reyes O S, et al.: Immunization using an Apo B-100 related epitope reduces atherosclerosis and plaque inflammation in hypercholesterolemic apo E (−/−) mice. Biochem Biophys Res Commun 2005; 338(4): 1982-9.
13. Fredrikson G N, Andersson L, Soderberg I, et al.: Atheroprotective immunization with MDA-modified apo B-100 peptide sequences is associated with activation of Th2 specific antibody expression. Autoimmunity 2005; 38(2): 171-9.
14. Fredrikson G N, Schiopu A, Berglund G, Alm R, Shah P K, Nilsson J: Autoantibody against the amino acid sequence 661-680 in apo B-100 is associated with decreased carotid stenosis and cardiovascular events. Atherosclerosis 2007; 194(2): e188-92.
15. Schiopu A, Bengtsson J, Soderberg I, et al.: Recombinant human antibodies against aldehyde-modified apolipoprotein B-100 peptide sequences inhibit atherosclerosis. Circulation 2004; 110(14): 2047-52.
16. Strom A, Fredrikson G N, Schiopu A, et al.: Inhibition of injury-induced arterial remodelling and carotid atherosclerosis by recombinant human antibodies against aldehyde-modified apoB-100. Atherosclerosis 2006; 190: 298-305.
17. Schiopu A, Frendeus B, Jansson B, et al.: Recombinant antibodies to an oxidized low-density lipoprotein epitope induce rapid regression of atherosclerosis in apobec-1(−/−)/low-density lipoprotein receptor(-/-) mice. J Am Coll Cardiol 2007; 50(24): 2313-8.

1. Complete oxidized LDL specific IgG fused or conjugated with at least one tissue stabilizing factor to be used in a medicine.

2. Complete oxidized LDL specific IgG fused or conjugated with at least one of the proteins of the group IL-10, TIMPs, and TGFβs to be used in a medicine

3. Complete oxidized LDL specific IgG according to claim 1, combined with IL-10 as fusion protein to be used in a medicine for treatment of atherosclerosis and prevention of clinical events in patients with atherosclerosis.

4. Complete oxidized LDL specific IgG according to claim 1, combined with TGFβ as fusion protein to be used in a medicine for treatment of atherosclerosis and prevention of clinical events in patients with atherosclerosis.

5. Complete oxidized LDL specific IgG according to claim 1 combined with TIMP as fusion protein to be used in a medicine for treatment of atherosclerosis and prevention of clinical events in patients with atherosclerosis.

6. Complete oxidized LDL specific IgG according to claim 1 combined with IL-10 as conjugated protein to be used in a medicine for treatment of atherosclerosis and prevention of clinical events in patients with atherosclerosis.

7. Complete oxidized LDL specific IgG according to claim 1 combined with TGFβ as conjugated protein to be used in a medicine for treatment of atherosclerosis and prevention of clinical events in patients with atherosclerosis.

8. Complete oxidized LDL specific IgG according to claim 1 combined with TIMP as conjugated protein to be used in a medicine for treatment of atherosclerosis and prevention of clinical events in patients with atherosclerosis.

9. Complete oxidized LDL specific IgG single chains according to claim 2 combined with IL-10 as fusion protein to be used in a medicine for treatment of atherosclerosis and prevention of clinical events in patients with atherosclerosis.

10. Complete oxidized LDL specific IgG Fab fragments according to claim 8 combined with IL-10 as fusion protein for treatment of atherosclerosis and prevention of clinical events in patients with atherosclerosis.

11. Complete oxidized LDL specific IgG according to claim 9 or 10 raised against the peptide with SEQ. ID. NO. 1, the peptide with SEQ. ID. NO. 2, the peptide with SEQ. ID. NO. 3, the peptide with SEQ. ID. NO. 4, the peptide with SEQ. ID. NO. 5, the peptide with SEQ. ID. NO. 6, the peptide with SEQ. ID. NO. 7, the peptide with SEQ. ID. NO. 8, the peptide with SEQ. ID. NO. 9, the peptide with SEQ. ID. NO. 10, the peptide with SEQ. ID. NO. 11, the peptide with SEQ. ID. NO. 12, the peptide with SEQ. ID. NO. 13, the peptide with SEQ. ID. NO. 14, the peptide with SEQ. ID. NO. 15, the peptide with SEQ. ID. NO. 16 and/or the peptide with SEQ. ID. NO. 17 for the treatment of atherosclerosis and prevention of clinical events in patients with atherosclerosis.

12. Complete oxidized LDL specific IgG according to claim 9 or 10, to be used in a fusion or conjugated protein in combination with IL-10 wherein the antibody comprises a variable heavy region (V_{H}) selected from the group of nucleic acid sequences consisting of the nucleic acid with SEQ. ID. NO. 101, the nucleic acid with SEQ. ID. NO. 103, the nucleic acid with SEQ. ID. NO. 105, the nucleic acid with SEQ. ID. NO. 107, the nucleic acid with SEQ. ID. NO. 109, the nucleic acid with SEQ. ID. NO. 111, the nucleic acid with SEQ. ID. NO. 113, the nucleic acid with SEQ. ID. NO. 115, the nucleic acid with SEQ. ID. NO. 117, the nucleic acid with SEQ. ID. NO. 119, the nucleic acid with SEQ. ID. NO. 121, the nucleic acid with SEQ. ID. NO. 123, the nucleic acid with SEQ. ID. NO. 125, the nucleic acid with SEQ. ID. NO. 127, the nucleic acid with SEQ. ID. NO. 129, and the nucleic acid with SEQ. ID. NO. 131.

13. Complete oxidized LDL specific IgG according to claim 12, wherein the antibody comprises a variable light region (V_{L}) selected from the group of nucleic acid sequences consisting of: the nucleic acid with SEQ. ID. NO. 102, the nucleic acid with SEQ. ID. NO. 104, the nucleic acid with SEQ. ID. NO. 106, the nucleic acid with SEQ. ID. NO. 108, the nucleic acid with SEQ. ID. NO. 110, the nucleic acid with SEQ. ID. NO. 112, the nucleic acid with SEQ. ID. NO. 114, the nucleic acid with SEQ. ID. NO. 116, the nucleic acid with SEQ. ID. NO. 118, the nucleic acid with SEQ. ID. NO. 120, the nucleic acid with SEQ. ID. NO. 122, the nucleic acid with SEQ. ID. NO. 124, the nucleic acid with SEQ. ID. NO. 126, the nucleic acid with SEQ. ID. NO. 128, the nucleic acid with SEQ. ID. NO. 130, and the nucleic acid with SEQ. ID. NO. 132.

14. Complete oxidized LDL specific IgG according to claim 12, wherein the antibody comprises a variable heavy region (V_{H}) selected from the group of nucleic acid sequences consisting of the nucleic acid with SEQ. ID. NO. 101, the nucleic acid with SEQ. ID. NO. 103, the nucleic acid with SEQ. ID. NO. 105, the nucleic acid with SEQ. ID. NO. 107, the nucleic acid with SEQ. ID. NO. 109, the nucleic acid with SEQ. ID. NO. 111, the nucleic acid with SEQ. ID. NO. 113, the nucleic acid with SEQ. ID. NO. 115, the nucleic acid with SEQ. ID. NO. 117, the nucleic acid with SEQ. ID. NO. 119, the nucleic acid with SEQ. ID. NO. 121, the nucleic acid with SEQ. ID. NO. 123, the nucleic acid with SEQ. ID. NO. 125, the nucleic acid with SEQ. ID. NO. 127, the nucleic acid with SEQ. ID. NO. 129, and the nucleic acid with SEQ. ID. NO. 131),
in combination with at least one variable light region (V_{L}) selected from the group of nucleic acid sequences consisting of the nucleic acid with SEQ. ID. NO. 102, the nucleic acid with SEQ. ID. NO. 104, the nucleic acid with SEQ. ID. NO. 106, the nucleic acid with SEQ. ID. NO. 108, the nucleic acid with SEQ. ID. NO. 110, the nucleic acid with SEQ. ID. NO. 112, the nucleic acid with SEQ. ID. NO. 114, the nucleic acid with SEQ. ID. NO. 116, the nucleic acid with SEQ. ID. NO. 118, the nucleic acid with SEQ. ID. NO. 120, the nucleic acid with SEQ. ID. NO. 122, the nucleic acid with SEQ. ID. NO. 124, the nucleic acid with SEQ. ID. NO. 126, the nucleic acid with SEQ. ID. NO. 128, the nucleic acid with SEQ. ID. NO. 130, and the nucleic acid with SEQ. ID. NO. 132.

15. Complete oxidized LDL specific IgG according to claim 12, wherein the antibody comprises a variable heavy region(V_{H}) with SEQ. ID. NO. 101 and a variable light region (V_{L}) with SEQ. ID. NO. 102.

16. Complete oxidized LDL specific IgG according to claim 12, wherein the antibody comprises a variable heavy region (V_{H}) with SEQ. ID. NO. 103 and a variable light region (V_{L}) with SEQ. ID. NO. 104.

17. Complete oxidized LDL specific IgG according to claim 12, wherein the antibody comprises a variable heavy region (V_{H}) with SEQ. ID. NO. 105 and a variable light region (V_{L}) with SEQ. ID. NO. 106).

18. Complete oxidized LDL specific IgG according to claim 12, wherein the antibody comprises a variable heavy region (V_{H}) with SEQ. ID. NO. 107 and a variable light region (V_{L}) with SEQ. ID. NO. 108.

19. Complete oxidized LDL specific IgG according to claim 12, wherein the antibody comprises a variable heavy region (V_{H}) with SEQ. ID. NO. 109 and a variable light region (V_{L}) with SEQ. ID. NO. 110.

20. Complete oxidized LDL specific IgG according to claim 12, wherein the antibody comprises a variable heavy region (V_{H}) with SEQ. ID. NO. 111 and a variable light region (V_{L}) with SEQ. ID. NO. 112.

21. Complete oxidized LDL specific IgG according to claim 12, wherein the antibody comprises a variable heavy region (V_{H}) with SEQ. ID. NO. 113 and a variable light region (V_{L}) with SEQ. ID. NO. 114.

22. Complete oxidized LDL specific IgG according to claim 12, wherein the antibody comprises a variable heavy region (V_{H}) with SEQ. ID. NO. 115 and a variable light region (V_{L}) with SEQ. ID. NO. 116.

23. Complete oxidized LDL specific IgG according to claim 12, wherein the antibody comprises a variable heavy region (V_{H}) with SEQ. ID. NO. 117 and a variable light region (V_{L}) with SEQ. ID. NO. 118.

24. Complete oxidized LDL specific IgG according to claim 12, wherein the antibody comprises a variable heavy region (V_{H}) with SEQ. ID. NO. 119 and a variable light region (V_{L}) with SEQ. ID. NO. 120.

25. Complete oxidized LDL specific IgG according to claim 12, wherein the antibody comprises a variable heavy region (V_{H}) with SEQ. ID. NO. 121 and a variable light region (V_{L}) with SEQ. ID. NO. 122.

26. Complete oxidized LDL specific IgG according to claim 12, wherein the antibody comprises a variable heavy region (V_{H}) with SEQ. ID. NO. 123 and a variable light region (V_{L}) with SEQ. ID. NO. 124.

27. Complete oxidized LDL specific IgG according to claim 12, wherein the antibody comprises a variable heavy region (V_{H}) with SEQ. ID. NO. 125 and a variable light region (V_{L}) with SEQ. ID. NO. 126.

28. Complete oxidized LDL specific IgG according to claim 12, wherein the antibody comprises a variable heavy region (V_{H}) with SEQ. ID. NO. 127 and a variable light region (V_{L}) with SEQ. ID. NO. 128.

29. Complete oxidized LDL specific IgG according to claim 12, wherein the antibody comprises a variable heavy region (V_{H}) with SEQ. ID. NO. 129 and a variable light region (V_{L}) with SEQ. ID. NO. 130.

30. Complete oxidized LDL specific IgG according to claim 12, wherein the antibody comprises a variable heavy region (V_{H}) with SEQ. ID. NO. 131 and a variable light region (V_{L}) with SEQ. ID. NO. 132.

31. Complete oxidized LDL specific IgG according to claim 12, wherein the antibody comprises a variable heavy region (V_{H}) with SEQ. ID. NO. 133 and a variable light region (V_{L}) with SEQ. ID. NO. 134.

32. Complete oxidized LDL specific IgG fused or conjugated with at least one tissue stabilizing factor.

33. Complete oxidized LDL specific IgG according to claim 32, fused or conjugated with at least one of the proteins of the group IL-10, TIMPs, and TGFβs.

34. Complete oxidized LDL specific IgG according to claim 32, combined with IL-10 as fusion protein.

35. Complete oxidized LDL specific IgG according to claim 32, combined with TGFβ as fusion protein.

36. Complete oxidized LDL specific IgG according to claim 32 combined with TIMP as fusion protein.

37. Complete oxidized LDL specific IgG according to claim 32 combined with IL-10 as conjugated protein.

38. Complete oxidized LDL specific IgG according to claim 32 combined with TGFβ as conjugated protein.

39. Complete oxidized LDL specific IgG according to claim 32 combined with TIMP as conjugated protein.

40. Complete oxidized LDL specific IgG single chains according to claim 33 combined with IL-10 as fusion protein.

41. Complete oxidized LDL specific IgG Fab fragments according to claim 40 combined with IL-10 as fusion protein.

42. Complete oxidized LDL specific IgG according to claim 40 or claim 41 raised against a peptide selected from the group consisting of the peptide with SEQ. ID. NO. 1, the peptide with SEQ. ID. NO. 2, the peptide with SEQ. ID. NO. 3. the peptide with SEQ. ID. NO. 4, the peptide with SEQ. ID. NO. 5, the peptide with SEQ. ID. NO. 6. the peptide with SEQ. ID. NO. 7, the peptide with SEQ. ID. NO. 8, the peptide with SEQ. ID. NO. 9, the peptide with SEQ. ID. NO. 10, the peptide with SEQ. ID. NO. 11, the peptide with SEQ. ID. NO. 12, the peptide with SEQ. ID. NO. 13, the peptide with SEQ. ID. NO. 14, the peptide with SEQ. ID. NO. 15, the peptide with SEQ. ID. NO. 16, and the peptide with SEQ. ID. NO. 17.

43. Complete oxidized LDL specific IgG according to claim 32, to be used in a fusion or conjugated protein in combination with IL-10 wherein the antibody comprises a variable heavy region (V_{H}) selected from the group of nucleic acid sequences consisting of the nucleic acid with SEQ. ID. NO. 101, the nucleic acid with SEQ. ID. NO. 103, the nucleic acid with SEQ. ID. NO. 105, the nucleic acid with SEQ. ID. NO. 107, the nucleic acid with SEQ. ID. NO. 109, the nucleic acid with SEQ. ID. NO. 111, the nucleic acid with SEQ. ID. NO. 113, the nucleic acid with SEQ. ID. NO. 115, the nucleic acid with SEQ. ID. NO. 117, the nucleic acid with SEQ. ID. NO. 119, the nucleic acid with SEQ. ID. NO. 121, the nucleic acid with SEQ. ID. NO. 123, the nucleic acid with SEQ. ID. NO. 125, the nucleic acid with SEQ. ID. NO. 127, the nucleic acid with SEQ. ID. NO. 129, and the nucleic acid with SEQ. ID. NO. 131.

44. Complete oxidized LDL specific IgG according to claim 32, wherein the antibody comprises a variable light region (V_{L}) selected from the group of nucleic acid sequences consisting of the nucleic acid with SEQ. ID. NO. 102, the nucleic acid with SEQ. ID. NO. 104, the nucleic acid with SEQ. ID. NO. 106, the nucleic acid with SEQ. ID. NO. 108, the nucleic acid with SEQ. ID. NO. 110, the nucleic acid with SEQ. ID. NO. 112, the nucleic acid with SEQ. ID. NO. 114, the nucleic acid with SEQ. ID. NO. 116, the nucleic acid with SEQ. ID. NO. 118, the nucleic acid with SEQ. ID. NO. 120, the nucleic acid with SEQ. ID. NO. 122, the nucleic acid with SEQ. ID. NO. 124, the nucleic acid with SEQ. ID. NO. 126, the nucleic acid with SEQ. ID. NO. 128, the nucleic acid with SEQ. ID. NO. 130 and the nucleic acid with SEQ. ID. NO. 132.

45. Pharmaceutical composition comprising complete oxidized LDL specific IgG fused or conjugated with at least one tissue stabilizing factor to be used in a medicine in combination with suitable adjuvants and excipients.

46. Pharmaceutical composition according to claim 45 comprising complete oxidized LDL specific IgG fused or conjugated with at least one of the proteins of the group IL-10, TIMPs, and TGFβs to be used in a medicine

47. Pharmaceutical composition according to claim 45 comprising complete oxidized LDL specific IgG combined with IL-10 as fusion protein to be used in a medicine for treatment of atherosclerosis and prevention of clinical events in patients with atherosclerosis.

48. Pharmaceutical composition according to claim 45 comprising complete oxidized LDL specific IgG combined with TGFβ as fusion protein to be used in a medicine for treatment of atherosclerosis and prevention of clinical events in patients with atherosclerosis.

49. Pharmaceutical composition according to claim 45 comprising complete oxidized LDL specific IgG according to claim 1 combined with TIMP as fusion protein to be used in a medicine for treatment of atherosclerosis and prevention of clinical events in patients with atherosclerosis.

50. Pharmaceutical composition according to claim 45 comprising complete oxidized LDL specific IgG combined with IL-10 as conjugated protein to be used in a medicine for treatment of atherosclerosis and prevention of clinical events in patients with atherosclerosis.

51. Pharmaceutical composition according to claim 45 comprising complete oxidized LDL specific IgG combined with TGFβ as conjugated protein to be used in a medicine for treatment of atherosclerosis and prevention of clinical events in patients with atherosclerosis.

52. Pharmaceutical composition according to claim 45 comprising complete oxidized LDL specific IgG combined with TIMP as conjugated protein to be used in a medicine for treatment of atherosclerosis and prevention of clinical events in patients with atherosclerosis.

53. Pharmaceutical composition according to claim 46 comprising complete oxidized LDL specific IgG single chains combined with IL-10 as fusion protein to be used in a medicine for treatment of atherosclerosis and prevention of clinical events in patients with atherosclerosis.

54. Pharmaceutical composition according to claim 53 comprising complete oxidized LDL specific IgG Fab fragments combined with IL-10 as fusion protein for treatment of atherosclerosis and prevention of clinical events in patients with atherosclerosis.

55. Pharmaceutical composition according to claim 52 or 53 comprising complete oxidized LDL specific IgG raised against a peptide selected from the group consisting of the peptide with SEQ. ID. NO. 1, the peptide with SEQ. ID. NO. 2, the peptide with SEQ. ID. NO. 3, the peptide with SEQ. ID. NO. 4, the peptide with SEQ. ID. NO. 5, the peptide with SEQ. ID. NO. 6, the peptide with SEQ. ID. NO. 7, the peptide with SEQ. ID. NO. 8, the peptide with SEQ. ID. NO. 9, the peptide with SEQ. ID. NO. 10, the peptide with SEQ. ID. NO. 11, the peptide with SEQ. ID. NO. 12, the peptide with SEQ. ID. NO. 13, the peptide with SEQ. ID. NO. 14, the peptide with SEQ. ID. NO. 15, the peptide with SEQ. ID. NO. 16 and the peptide with SEQ. ID. NO. 17.

56. Method for treating atherosclerosis and prevention of clinical events in patients with atherosclerosis wherein a therapeutically effective amount of a complete oxidized LDL specific IgG fused or conjugated with at least one tissue stabilizing factor is administered to a patient suffering from atherosclerosis.


SEQUENCE LISTING (49 sequences)

SEQ ID NO: 1 - protein, 20 aa, Homo sapiens
FLDTVYGNCSTHFTVKTRKG

SEQ ID NO: 2 - protein, 20 aa, Homo sapiens
PQCSTHILQWLKRVHANPLL

SEQ ID NO: 3 - protein, 20 aa, Homo sapiens
VISIPRLQAEARSEILAHWS

SEQ ID NO: 4 - protein, 20 aa, Homo sapiens
IALDDAKINFNEKLSQLQTY

SEQ ID NO: 5 - protein, 20 aa, Homo sapiens
KTTKQSFDLSVKAQYKKNKH

SEQ ID NO: 6 - protein, 20 aa, Homo sapiens
EEEMLENVSLVCPKDATRFK

SEQ ID NO: 7 - protein, 20 aa, Homo sapiens
GSTSHHLVSRKSISAALEHK

SEQ ID NO: 8 - protein, 20 aa, Homo sapiens
IENIDFNKSGSSTASWIQNV

SEQ ID NO: 9 - protein, 20 aa, Homo sapiens
IREVTQRLNGEIQALELPQK

SEQ ID NO: 10 - protein, 20 aa, Homo sapiens
EVDVLTKYSQPEDSLIPFFE

SEQ ID NO: 11 - protein, 20 aa, Homo sapiens
ALLVPPETEEAKQVLFLDTV

SEQ ID NO: 12 - protein, 20 aa, Homo sapiens
IEIGLEGKGFEPTLEALFGK

SEQ ID NO: 13 - protein, 20 aa, Homo sapiens
SGASMKLTTNGRFREHNAKF

SEQ ID NO: 14 - protein, 20 aa, Homo sapiens
NLIGDFEVAEKINAFRAKVH

SEQ ID NO: 15 - protein, 20 aa, Homo sapiens
GHSVLTAKGMALFGEGKAEF

SEQ ID NO: 16 - protein, 20 aa, Homo sapiens
FKSSVITLNTNAELFNQSDI

SEQ ID NO: 17 - protein, 20 aa, Homo sapiens
FPDLGQEVALNANTKNQKIR

SEQ ID NO: 18 - DNA, 369 bp, Artificial Sequence
  Antibody against human apoB100 peptide
gaggtgcagctgttggagtctgggggaggcttggtacagcctggggggtccctgagactctcctgtgcagcctctggattcaccttcaataacgcctggatgagctgggtccgccaggctccagggaaggggctggagtgggtctcatccattagtagtagtagtagttacatatactacgcagactcagtgaagggccgattcaccatctccagagacaattccaagaacacgctgtatctgcaaatgaacagcctgagagccgaggacactgccgtgtattactgtgcgagagtcagtaggtactactacggaccatctttctactttgactcctggggccagggtacactggtcaccgtgagcagc

SEQ ID NO: 19 - DNA, 336 bp, Artificial Sequence
  Antibody against human apoB100 peptide
cagtctgtgctgactcagccaccctcagcgtctgggacccccgggcagagggtcaccatctcctgctctggaagcaggtccaacattgggaataattatgtatcctggtatcagcagctcccaggaacggcccccaaactcctcatctatggtaacaacaatcggccctcaggggtccctgaccgattctctggctccaagtctggcacctcagcctccctggccatcagtgggctccggtccgaggatgaggctgattattactgtgcagcatgggatgacagcctgaatggtcattgggtgttcggcggaggaaccaagctgacggtcctaggt

SEQ ID NO: 20 - DNA, 366 bp, Artificial Sequence
  Antibody against human apoB100 peptide
gaggtgcagctgttggagtctgggggaggcttggtacagcctggggggtccctgagactctcctgtgcggcctctggattcaccttcagtgactactacatgagctgggtccgccaggctcccgggaaggggctggagtgggtatcgggtgttagttggaatggcagtaggacgcactatgcagactctgtgaagggccgattcaccatctccagagacaattccaagaacacgctgtatctgcaaatgaacagcctgagagccgaggacactgccgtgtattactgtgcgagagcggctaggtactcctactactactacggtatggacgtctggggccaaggtacactggtcaccgtgagcagc

SEQ ID NO: 21 - DNA, 327 bp, Artificial Sequence
  Antibody against human apoB100 peptide
cagtctgtgctgactcagccaccctcagcgtctgggacccccgggcagagggtcaccatctcctgttctggaagcagctccaacatcggaaataatgctgtaaactggtatcagcagctcccaggaacggcccccaaactcctcatctatgggaatgatcggcggccctcaggggtccctgaccgattctctggctccaagtctggcacctcagcctccctggccatcagtgggctccggtccgaggatgaggctgattattactgtcagacctggggcactggccggggggtattcggcggaggaaccaagctgacggtcctaggt

SEQ ID NO: 22 - DNA, 366 bp, Homo sapiens
gaggtgcagctgttggagtctgggggaggcttggtacagcctggggggtccctgagactctcctgtgcagcctctggattcacctttagtagctattggatgagctgggtccgccaggctccagggaaggggctggagtgggtctcaagtatcagtggtagtggtcgtaggacatactacgcagactccgtgcagggccggttcaccatctccagagacaattccaagaacacgctgtatctgcaaatgaacagcctgagagccgaggacactgccgtgtattactgtgcgagattggtctcctatggttcggggagtttcggttttgactactggggccaaggtacactggtcaccgtgagcagc

SEQ ID NO: 23 - DNA, 333 bp, Artificial Sequence
  Antibody against human apoB100 peptide
cagtctgtgctgactcagccaccctcagcgtctgggacccccgggcagagggtcaccatctcttgttctggaagcagctccaatatcggaagtaattatgtatcctggtatcagcagctcccaggaacggcccccaaactcctcatctatggtaactacaatcggccctcaggggtccctgaccgattctctggctccaagtctggcacctcagcctccctggccatcagtgggctccggtccgaggatgaggctgattattactgtgcagcatgggatgacagcctgagtggttgggtgttcggcggaggaaccaagctgacggtcctaggt

SEQ ID NO: 24 - DNA, 378 bp, Artificial Sequence
  Antibody against human apoB100 peptide
gaggtgcagctgttggagtctgggggaggcttggtacagcctggggggtccctgagactctcctgtgcagcctctggattcaccttcagtaacgcctggatgagctgggtccgccaggttccagggaaggggctggagtgggtctcaactcttggtggtagtggtggtggtagcacatactacgcagactccgtgaagggccggttcaccatctccagagacaattccaagaacacgctgtatctgcaaatgaacagcctgagagccgaggacactgccgtgtattactgtgcgaagttaggggggcgatcccgatatgggcggtggccccgccaatttgactactggggccaaggtacactggtcaccgtgagcagc

SEQ ID NO: 25 - DNA, 333 bp, Artificial Sequence
  Antibody against human apoB100 peptide
cagtctgtgctgactcagccaccctcagcgtctgggacccccgggcagagggtcaccatctcctgctctggaagcagctccaacattggaaataactatgtatcctggtatcagcagctcccaggaacggcccccaaactcctcatctatagtaataatcagcggccctcaggggtccctgaccgattctctggctccaagtctggcacctcagcctccctggccatcagtgggctccggtccgaggatgaggctgattattactgtgcagcatgggatgacagcctgagtcattggctgttcggcggaggaaccaagctgacggtcctaggt

SEQ ID NO: 26 - DNA, 372 bp, Artificial Sequence
  Antibody against human apoB100 peptide
gaggtgcagctgttggagtctgggggaggcttggtacagcctggggggtccctgagactctcctgtgcagcctctggattcaccttcagtgactactacatgagctggatccgccaggctccagggaaggggctggagtgggtctcaagtatcagtggccgtgggggtagttcctactacgcagactccgtgaggggccggttcaccatctccagagacaattccaagaacacgctgtatctgcaaatgaacagcctgagagccgaggacactgccgtgtattactgtgcgagactttcctacagctatggttacgagggggcctactactttgactactggggccagggtacactggtcaccgtgagcagc

SEQ ID NO: 27 - DNA, 333 bp, Homo sapiens
cagtctgtgctgactcagccaccctcagcgtctgggacccccgggcagagggtcaccatctcctgctctggaagcagctccaacattgggaataattatgtatcctggtatcagcagctcccaggaacggcccccaaactcctcatctataggaataatcagcggccctcaggggtccctgaccgattctctggctccaagtctggcaccttagcctccctggccatcagtgggctccggtccgaggatgaggctgattattactgtgcaacctgggatgacagcctgaatggttgggtgttcggcggaggaaccaagctgacggtcctaggt

SEQ ID NO: 28 - DNA, 363 bp, Artificial Sequence
  Antibody against human apoB100 peptide
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcatccattagtagtagtggtcgtttcatttactacgcagactcaatgaagggccgcttcaccatctccagagacaattccaagaacacgctgtatctgcaaatgaacagcctgagagccgaggacactgccgtgtattactgtacgaggctccggagagggagctacttctgggcttttgatatctggggccaaggtacactggtcaccgtgagcagc

SEQ ID NO: 29 - DNA, 333 bp, Artificial Sequence
  Antibody against human apoB100 peptide
cagtctgtgctgactcagccaccctcagcgtctgggacccccgggcagagggtcaccatctcctgttctggaagcagctccaacattggcggtgagtctgtatcctggtatcagcagctcccaggaacggcccccaaactcctcatctatagtaataatcagcggccctcaggggtccctgaccgattctctggctccaagtctggcacctcagcctccctggccatcagtgggctccggtccgaggatgaggctgattattactgtgcagcatgggatgacagcctgaatggttgggtgttcggcggaggaaccaagctgacggtcctaggt

SEQ ID NO: 30 - DNA, 369 bp, Artificial Sequence
  Antibody against human apoB100 peptide
gaggtgcagctgttggagtctgggggaggcttggtacagcctggggggtccctgagactctcctgtgcagcctctggattcacctttagaacgtattggatgacctgggtccgccaggctccagggaaggggctggagtgggtctcatctattagcagtagcagtaattacatattctacgcagactcagtgaagggccgattcaccatctccagagacaattccaagaacacgctgtatctgcaaatgaacagcctgagagccgaggacactgccgtgtattactgtgcgagactcagacggagcagctggtacggggggtactggttcgacccctggggccaaggtacactggtcaccgtgagctca

SEQ ID NO: 31 - DNA, 335 bp, Artificial Sequence
  Antibody against human apoB100 peptide
cagtctgtgctgactcagccaccctcagcgtctgggacccccgggcagagggtcaccatctcctgctctggaagcagctccaacattgggaataattatgtatcctggtatcagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgaatggtattgggtgttcggcggaggaaccaagctgacggtcctaggt

SEQ ID NO: 32 - DNA, 369 bp, Artificial Sequence
  Antibody against human apoB100 peptide
gaggtgcagctgttggagtctgggggaggcttggtacagcctggggggtccctgagactctcctgtgcagcctctggattcaccttcagtagcaactacatgagctgggtccgccaggctccagggaaggggctggagtgggtctcatccattagtagtagtagtagttacatatactacgcagactcagtgaagggccgattcaccatctccagagacaattccaagaacacgctgtatctgcaaatgaacagcctgagagccgaggacactgccgtgtattactgtgcgagagtaggccggtataactggaagacggggcatgcttttgatatctggggccagggtacactggtcaccgtgagctca

SEQ ID NO: 33 - DNA, 333 bp, Artificial Sequence
  Antibody against human apoB100 peptide
cagtctgtgctgactcagccaccctcagcgtctgggacccccgggcagagggtcaccatctcctgctctggaaggacctacaacattggaaataattatgtatcgtggtatcagcagctcccaggaacggcccccaaactcctcatctatggtaacatcaatcggccctcaggggtccctgaccgattctctggctccaagtctggcacctcagcctccctggccatcagtgggctccggtccgaggatgaggctgattattactgtgcagcatgggatgtcaggctgaatggttgggtgttcggcggaggaaccaagctgacggtcctaggt

SEQ ID NO: 34 - DNA, 381 bp, Artificial Sequence
  Antibody against human apoB100 peptide
gaggtgcagctgttggagtctgggggaggcttggtacagcctggggggtccctgagactctcctgtgcagcctctggattcaccttccgtgactactacgtgagctggatccgccaggctccagggaaggggctggagtgggtctcaagtattagtggtagtgggggtaggacatactacgcagactccgtggagggccggttcaccatctccagagacaattccaagaacacgctgtatctgcaaatgaacagcctgagagccgaggacactgccatgtattactgtgccagagtatccgcccttcggagacccatgactacagtaactacttactggttcgacccctggggccaaggtacactggtcaccgtgagctca

SEQ ID NO: 35 - DNA, 333 bp, Artificial Sequence
  Antibody against human apoB100 peptide
cagtctgtgctgactcagccaccctcagcgtctgggacccccgggcagagggtcaccatctcctgctctggaaggagctccaacattgggaatagttatgtctcctggtatcagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcaggatgggatgacaccctgcgtgcttgggtgttcggcggaggaaccaagctgacggtcctaggt

SEQ ID NO: 36 - DNA, 360 bp, Artificial Sequence
  Antibody against human apoB100 peptide
gaggtgcagctgttggagtctgggggaggcttggtacagcctggggggtccctgagactctcctgtgcagcctctggattcaccttcagtaacgcctggatgagctgggtccgccaggctccagggaaggggctggagtgggtctccgctattagtggtagtggtaacacatactatgcagactccgtgaagggccggttcaccatctccagagacaattccaagaacacgctgtatctgcaaatgaacagcctgagagccgaggacactgccgtgtattactgtgcgagagcctcccaccgtatattaggttatgcttttgatatctggggccagggtacactggtcaccgtgagctca

SEQ ID NO: 37 - DNA, 328 bp, Artificial Sequence
  Antibody against human apoB100 peptide
cagtctgtgctgactcagccaccctcagcgtctgggacccccgggcagagggtcaccatctcttgttctggaagccgctccaacatcgggagaaatgctgttagttggtatcagcagctcccaggaacggcccccaaactcctcatctatgctaacagcaatcggccctcaggggtccctgaccgattctctggctccaagtctggcacctcagcctccctggccatcagtgggctccggtccgaggatgaggctgattattactgtgcagcatgggatggcagcctgaatggttgggtgttcggcggaggaaccaagctgacggtcc

SEQ ID NO: 38 - DNA, 363 bp, Artificial Sequence
  Antibody against human apoB100 peptide
gaggtgcagctgttggagtctgggggaggcttggtacagcctggggggtccctgagactctcctgtgcagcctctggattcaccttcagtaacgcctggatgagctgggtccgccaggctccagggaaggggctggagtgggtctcaagtattagtgttggtggacataggacatattatgcagattccgtgaagggccggtccaccatctccagagacaattccaagaacacgctgtatctgcaaatgaacagcctgagagccgaggacactgccgtgtattactgtgcacggatacgggtgggtccgtccggcggggcctttgactactggggccagggtacactggtcaccgtgagctca

SEQ ID NO: 39 - DNA, 333 bp, Artificial Sequence
  Antibody against human apoB100 peptide
cagtctgtgctgactcagccaccctcagcgtctgggacccccgggcagagggtcaccatctcctgctctggaagcaacaccaacattgggaagaactatgtatcttggtatcagcagctcccaggaacggcccccaaactcctcatctatgctaatagcaatcggccctcaggggtccctgaccgattctctggctccaagtctggcacctcagcctccctggccatcagtgggctccggtccgaggatgaggctgattattactgtgcgtcatgggatgccagcctgaatggttgggtattcggcggaggaaccaagctgacggtcctaggt

SEQ ID NO: 40 - DNA, 378 bp, Homo sapiens
gaggtgcagctgttggagtctgggggaggcttggtacagcctggggggtccctgagactctcctgtgcagcctctggattcaccttcagtaacgcctggatgagctgggtccgccaggctccagggaaggggctggagtgggtctcatccattagtagtagtagtagttacatatactacgcagactcagtgaagggccgatccaccatctccagagacaattccaagaacacgctgtatctgcaaatgaacagcctgagagccgaggacactgccgtgtattactgtgcgaggctcacaaatattttgactggttattatacctcaggatatgcttttgatatctggggccaaggtacactggtcaccgtgagctca

SEQ ID NO: 41 - DNA, 333 bp, Artificial Sequence
  Antibody against human apoB100 peptide
cagtctgtgctgactcagccaccctcagcgtctgggacccccgggcagagggtcaccatctcctgctctggaagcacctccaacattgggaagaattatgtatcctggtatcagcagctcccaggaacggcccccaaactcctcatctatggtaacagcaatcggccctcaggggtccctgaccgattctctggctccaagtctggcacctcagcctccctggccatcagtgggctccggtccgaggatgaggctgattattactgtgcagcatgggatgccagcctcagtggttgggtgttcggcggaggaaccaagctgacggtcctaggt

SEQ ID NO: 42 - DNA, 363 bp, Artificial Sequence
  Antibody against human apoB100 peptide
gaggtgcagctgttggagtctgggggaggcttggtacagcctggggggtccctgagactctcctgtgcagcctctggattcaccttcagtagttcttggatgagttgggtccgccaggctccagggaaggggctggagtgggtctcatccattagtagtagtagtagttacatatactacgcagactcagtgaagggccgattcaccatctccagagacaattccaagaacacgctgtatctgcaaatgaacagcctgagagccgaggacactgccgtgtattactgtgcgagagtagggaactacggtttctaccactacatggacgtctggggccaaggtacactggtcaccgtgagctca

SEQ ID NO: 43 - DNA, 333 bp, Artificial Sequence
  Antibody against human apoB100 peptide
cagtctgtgctgactcagccaccctcagcgtctgggacccccgggcagagggtcaccatctcttgttctggaggcagctcaaacatcggaaaaagaggtgtaaattggtatcagcagctcccaggaacggcccccaaactcctcatctatggtaacagaaatcggccctcaggggtccctgaccgattctctggctccaagtctggcacctcagcctccctggccatcagtgggctccggtccgaggatgaggctgattattactgtgctacatgggattacagcctcaatgcttgggtgttcggcggaggaaccaagctgacggtcctaggt

SEQ ID NO: 44 - DNA, 366 bp, Artificial Sequence
  Antibody against human apoB100 peptide
gaggtgcagctgttggagtctgggggaggcttggtacagcctggggggtccctgagactctcctgtgcagcctctggattcacctttagtagctattggatgagctgggtccgccaggctccagggaaggggctggagtgggtctcatccattagtagtagtagtagttacatatactacgcagactcagtgaagggccgattcaccatctccagagacaattccaagaacacgctgtatctgcaaatgaacagcctgagagccgaggacactgccgtgtattactgtgcgagaattaaacggttacgattcggctggaccccttttgactactggggccagggtacactggtcaccgtgagctca

SEQ ID NO: 45 - DNA, 333 bp, Artificial Sequence
  Antibody against human apoB100 peptide
cagtctgttctgactcagccaccctcagcgtctgggacccccgggcagagggtcaccatctcctgttctggaagcagctccaacatcggaaataatggtgtaaactggtatcagcagctcccaggaacggcccccaaactcctcatctatggtaacaacaatcggccctcaggggtccctgaccgattctctggctccaagtctggcacctcagcctccctggccatcagtgggctccggtccgaggatgaggctgattattactgtgcagcatgggatgacagcctgcgtggttggctgttcggcggaggaaccaagctgacggtcctaggt

SEQ ID NO: 46 - DNA, 309 bp, Artificial Sequence
  Antibody against human apoB100 peptide
tcctgtgcagcctctggattcaccttcagtaacgcctggatgagctgggtccgccaggctccagggaaggggctggagtgggtctcatccattagtagtagtagtagttacatatactacgcagactcagtgaagggccgattcaccatctccagagacaattccaagaacacgctgtatctgcaaatgaacagcctgagagccgaggacactgccgtgtattactgtgcgagagtcaatagcaaaaagtggtatgagggctacttctttgactactggggccagggtacactggtcaccgtgagctca

SEQ ID NO: 47 - DNA, 333 bp, Artificial Sequence
  Antibody against human apoB100 peptide
cagtctgtgctgactcagccaccctcagcgtctgggacccccgggcagagggtcaccatctcctgctctggaagcagctccaacattgggaataattatgtatcctggtatcagcagctcccaggaacggcccccaaactcctcatctatggtaacagcaatcggccctcaggggtccctgaccgattctctggctccaagtctggcacctcagcctccctggccatcagtgggctccggtccgaggatgaggctgattattactgtgcagcatgggatgacagtctgagtggttgggtgttcggcggaggaaccaagctgacggtcctaggt

SEQ ID NO: 48 - DNA, 375 bp, Artificial Sequence
  Antibody against human apoB100 peptide
gaggtgcagctgttggagtctgggggaggcttggtacagcctggggggtccctgagactctcctgtgcagcctctggattcaccttcagtaacgcctggatgagctgggtccgccaggctccagggaaggggctggagtgggtctcatccattagtactagtagtaattacatatactacgcagactcagtgaagggccggttcaccatctccagagacaattccaagaacacgctgtatctgcaaatgaacagcctgagagccgaggacactgccgtgtattactgtgcgagagtcaagaagtatagcagtggctggtactcgaattatgcttttgatatctggggccaaggtacactggtcaccgtgagctca

SEQ ID NO: 49 - DNA, 333 bp, Artificial Sequence
  Antibody against human apoB100 peptide
cagtctgtgctgactcagccaccctcagcgtctgggacccccgggcagagggtcaccatctcctgctctggaagcagctccagcattgggaataattttgtatcctggtatcagcagctcccaggaacggcccccaaactcctcatctatgacaataataagcgaccctcaggggtccctgaccgattctctggctccaagtctggcacctcagcctccctggccatcagtgggctccggtccgaggatgaggctgattattactgtgcagcatgggatgacagcctgaatggttgggtgttcggcggaggaaccaagctgacggtcctaggt